(19) United States
(12) Patent Application Publication
(10) Pub. No.: US 2020/0046241 A1
(43) Pub. Date: Feb. 13, 2020

(54) ECG AND PCG MONITORING SYSTEM FOR DETECTION OF HEART ANOMALY

(71) Applicants: NANYANG POLYTECHNIC, Singapore (SG); Singapore Health Services Pte Ltd, Singapore (SG)

(72) Inventors: Kwai Fern LAM, Singapore (SG); Chin Tiong NG, Singapore (SG); Jia Xin LOW, Singapore (SG); Chee Teck PHUA, Singapore (SG); Jiunn Liang Jonathan YAP, Singapore (SG); Swee Yaw TAN, Singapore (SG); En Hou Philip WONG, Singapore (SG)

(73) Assignees: NANYANG POLYTECHNIC, Singapore (SG); Singapore Health Services Pte Ltd, Singapore (SG)

(21) Appl. No.: 16/500,985

(22) PCT Filed: Apr. 6, 2018
(86) PCT No.: PCT/SG2018/050176

§ 371 Date: Oct. 4, 2019

(30) Foreign Application Priority Data
Apr. 7, 2017 (SG) 10201702899P

(51) Int. Cl. A61B 5/0402; A61B 7/04

(52) U.S. Cl. CPC A61B 5/0402; A61B 7/04; A61B 2562/0204; A61B 2560/0214; A61B 2562/16

(57) ABSTRACT

This invention relates to a system and a method for continuous monitoring of the heart activities via a mobile device and algorithm to detect heart anomalies based on readings on electrocardiogram (ECG) and phonocardiogram (PCG). The system includes an integrated ECG and PCG apparatus comprising: a housing having a top part and a bottom part, the bottom part of the housing includes a tapered surface extending from a perimeter of a bottom surface to an opening at the top of the bottom part forming an acoustic chamber; a power source housed within the top part; an audio receiver arranged to seal the opening for obtaining PCG signal; a plurality of dry sensors arranged at the bottom surface for obtaining ECG signal; a processing unit powered by the power source and communicatively connectable to the audio receiver and the plurality of dry sensors, wherein the bottom part of the housing is adaptably configured to create a snug fit on a subject preventing the audio receiver from picking acoustic noise from outside the acoustic chamber.

### FIELD OF THE INVENTION

This invention relates to a system and a method for continuous monitoring of the heart activities via a mobile device and algorithm to detect heart anomalies based on readings on electrocardiogram (ECG) and phonocardiogram (PCG).

### PRIOR ART

Heart failure is a global public health issue of epidemic proportions and represents a tremendous burden to overall healthcare costs. At least 5 million Americans have heart failure and approximately 550,000 new cases are diagnosed each year in the US alone. In fact, heart disease accounts for 30% of death worldwide and in years to come will continue to be the leading cause of morbidity and mortality worldwide. One of the first steps in evaluating the heart system after detailed history taking is physical examination. Auscultation of the heart or listening to the heart sound forms the core of heart physical examination. Heart auscultation provides important initial clues in patient evaluation and serves as a guide for further diagnostic testing.

Listening to the heart sound forms the core of heart physical examination for diagnosis of heart disease. Doctors who are experienced can perform diagnosis of an abnormal heart through the use of stethoscope.

Heart sounds are generated by the vibrations from the different chambers of the heart. The main normal heart sounds are the S1 and the S2 heart sound. The S3 heart sound can, at times be innocent but may be pathologic; caused by disease. An S4 heart sound is almost always pathologic. Heart sounds can be complex and only experienced doctors are able to differentiate them using their intensity, pitch, location, quality and timing in the cardiac cycle.

### SUMMARY OF THE INVENTION

The above and other problems are solved and an advance in the state of the art is made by a system and method provided by embodiments in accordance with this invention. A first advantage of embodiments of systems and methods in accordance with the disclosure is that patients with suspected heart disease can be monitored remotely. This means that patients are not required to be warded for health monitoring. A second advantage of embodiments of systems and methods in accordance with the disclosure is that heart condition can be monitored continuously to pick up abnormal heart sound signals. Importantly, the occurrence of heart disease is non-regular or sporadic. Hence, it is advantageous to monitor a patient with risk of heart disease for prolong period. A third advantage of embodiments of systems and methods in accordance with the disclosure is that the system improves pre-diagnosis by highlighting to doctors of patient having early clues or signs of heart disease.

A first aspect of the disclosure describes an integrated electrocardiogram (ECG) and phonocardiogram (PCG) apparatus. The apparatus comprises: a housing having a top part and a bottom part, the bottom part of the housing includes a tapered surface extending from a perimeter of a bottom surface to an opening at the top of the bottom part forming an acoustic chamber; a power source housed within the top part; an audio receiver arranged to seal the opening for obtaining PCG signal; a number of dry sensors arranged at the bottom surface for obtaining ECG signal; a processing unit powered by the power source and communicatively connectable to the audio receiver and the dry sensors, wherein the bottom part of the housing is adaptably configured to create a snug fit on a subject preventing the audio receiver from picking acoustic noise from outside the acoustic chamber.

In an embodiment of the first aspect of the disclosure, the audio receiver is an electret microphone which covers frequencies of 20 Hz˜20 kHz.

In an embodiment of the first aspect of the disclosure, the apparatus further comprise another dry sensor arranged at the top part of the housing and in parallel connection with one of the plurality of dry sensor.

In an embodiment of the first aspect of the disclosure, the apparatus further comprises a pair of attachment rings on a side surface of the housing.

In an embodiment of the first aspect of the disclosure, the apparatus further comprises a touch sensor for activating the processing unit. The touch sensor is skin resistance based sensor.

In an embodiment of the first aspect of the disclosure, the processing unit comprises: a processor, memory, transceiver, and instructions stored on the memory and executable by the processor to: receive signals from dry sensors and audio receiver and store the signals from the dry sensors as ECG signals and the signals from the audio receiver as PCG signals in the memory; receive a request to connect via the transceiver and in response, attempt to connect to a requestor; and transmit the ECG and PCG signals stored on the memory to the requestor upon successful connection with the requestor.

In an embodiment of the first aspect of the disclosure, the processing unit comprises: a processor, memory, transceiver, and instructions stored on the memory and executable by the processor to: receive signal from the touch sensor and in response, initiate collection of ECG and PCG signals from the dry sensors and audio receiver respectively; receive signals from dry sensors and audio receiver and store the signals from the dry sensors as ECG signals and the signals from the audio receiver as PCG signals in the memory; receive a request to connect via the transceiver and in response, attempt to connect to a requestor; and transmit the ECG and PCG signals stored on the memory to the requestor upon successful connection with the requestor.

A second aspect of the disclosure describes a heart monitoring system comprising: the integrated ECG and PCG apparatus according to that described above in relation to the first aspect of the disclosure, and a processing unit comprising a processor, memory and instructions stored on the memory and executable by the processor to: receive the signal from the integrated ECG and PCG apparatus; apply a low pass filter to each of the ECG and PCG signals; process the filtered ECG signal to obtain a start point (SP) and an end point (EP); select a region between the SP and EP of the filtered PCG signal and analyse the PCG discrete signal of the selected region to determine a first segment, a second segment, a third segment and a fourth segment.

In an embodiment of the second aspect of the disclosure, the instruction to process the filtered ECG signal to obtain a start point (SP) and an end point (EP) comprises instructions to: apply a wavelet decomposition to the filtered ECG signal and zeroing all coefficients other than the selected level value (e.g. 5^{th} level value; value depends on the sampling frequency); apply a wavelet reconstruction to resynthesize the signal; determine the R peaks of the ECG signal by taking the absolute of the square values; shift the first and second R peaks value left by a predetermined range; and assign the shifted first R peak value as the SP and the shifted second R peak value as the EP.

In an embodiment of the second aspect of the disclosure, the instruction to analyse the PCG discrete signal of the selected region to determine a first segment, a second segment, a third segment and a fourth segment comprises instructions to: apply a wavelet decomposition to the filtered ECG signal and zeroing all coefficients other than the selected level value; apply a wavelet reconstruction to resynthesize the signal; determine the S peaks of the PCG signal by taking the absolute of the square values; and identify the first, second, third and fourth segments of a cardiac cycle, which includes the first heart sound (S1) and second heart sound (S2), based on the detected S peaks of the PCG signal.

In an embodiment of the second aspect of the disclosure, the instruction to identify the first, second, third and fourth segments of a cardiac cycle are based on the detected S peaks of the PCG signal comprises instructions to: identify the first segment from −50 ms of the first S peak to +50 ms of the first S peak, the first segment containing the first heart sound (S1); identify the third segment from −30 ms of the second S peak to +30 ms of the second S peak, the third segment containing the second heart sound (S2); identify the second segment from +50 ms of the first S peak to −30 ms of the second S peak; and identify the fourth segment from +30 ms of the second S peak to end of selected region.

In an embodiment of the second aspect of the disclosure, the processing unit further comprises instructions to: determine heart sound anomaly based on the second and fourth segments; and classify as abnormal in response to determining heart sound anomaly.

In an embodiment of the second aspect of the disclosure, the instruction to determine heart sound anomaly based on the second segment comprises instructions to: calculate frequency and energy from the second segment; compare the frequency and energy with a predetermined threshold; and classify as abnormal in response to the frequency and energy being above the predetermined threshold.

In an embodiment of the second aspect of the disclosure, the instruction to determine heart sound anomaly based on fourth segment comprises instructions to: calculate frequency and energy from fourth segment; compare the frequency and energy with a predetermined threshold; and classify as abnormal in response to the frequency and energy being above the predetermined threshold.

### BRIEF DESCRIPTION OF THE DRAWINGS

The above and other features and advantages in accordance with this invention are described in the following detailed description and are shown in the following drawings:

FIG. 1 illustrating a system architecture in accordance with this disclosure;

FIG. 2 illustrating various perspective views of an exemplary design of the apparatus in accordance with this disclosure;

FIG. 3 illustrating a cross sectional view of the exemplary design of the apparatus in accordance with this disclosure;

FIG. 4 illustrating a cross-sectional view between the line A-A as shown in FIG. 3 of the bottom view of the apparatus in accordance with this disclosure;

FIG. 5 illustrating a bottom view of the apparatus in accordance with this disclosure;

FIG. 6 illustrating a top view of the apparatus in accordance with this disclosure;

FIG. 7 illustrating a right side view of the apparatus in accordance with this disclosure;

FIG. 8 illustrating an exemplary electronic circuit design for ECG signal acquisition that is implemented in the apparatus in accordance with this disclosure;

FIG. 9 illustrating an ECG waveform with dry sensor connected to point 3 of the apparatus in accordance with this disclosure;

FIG. 10 illustrating an ECG waveform without dry sensor connected to point 3 of the apparatus in accordance with this disclosure;

FIG. 11 illustrating an exemplary electronic circuit design for touch sensor that is implemented in the apparatus in accordance with this disclosure;

FIG. 12 illustrating a block diagram of components in a processing unit of the apparatus in accordance with this disclosure;

FIG. 13 illustrates an example of a processing system in a mobile device or cloud server that performs processes in accordance with an embodiment of this disclosure;

FIG. 14 illustrating the positions of stethoscope to determine heart sound;

FIG. 15 illustrating one possible position of the apparatus being placed on a chest of a user in accordance with this disclosure;

FIG. 16 illustrating exemplary waveform acquired for signal processing in accordance with this disclosure;

FIG. 17.1 illustrating a flowchart of S1, S2 and Murmur identification in accordance with this disclosure;

FIGS. 17.2-17.4 illustrating various a blow up views of the flowchart as shown in FIG. 17.1; and

FIG. 18 illustrating a process flow performed by the cloud server to process the ECG and PCG signals for each patient.

### DETAILED DESCRIPTION

This invention relates to a system and a method for continuous monitoring of the heart activities via a mobile device and algorithm to detect heart anomalies based on readings on electrocardiogram (ECG) and phonocardiogram (PCG).

Some abnormality from the heart sound occurs sporadically or at irregular intervals. As such it might not be able to capture or heard by the physician during the time of diagnosis. The process of diagnosis using stethoscope is performed at different location of the human chest to identify or categorize the different heart sound (S1, S2, S3 or S4). This process is difficult to automate. Further, there is no means of monitoring heart sound as experienced doctor are required to listen to the heart sound.

These challenges not only hinder diagnosis but also hinder telehealth deployment to monitor patients with heart disease remotely or at home. This has in turn burdened the healthcare system as every year many people are hospitalized for check-up on heart related disease alone.

This disclosure provides an apparatus that is configured to continuously acquire and monitor heart sound; and process and classify the heart condition using heart sound.

The apparatus is relatively small (about the size of the normal stethoscope), easy to use and cost-effective such that it can be deployed at home or anywhere which is convenient to the patients or users.

Briefly, the apparatus combines both stethoscope and ECG capability while compacted into a small wearable device for the user. ECG essentially provides the timing information to accurately determine the heart sound (S1, S2 and S3/S4 if any). As such the heart sound measurement can be done at any single location on the chest (as opposed to multiple specific locations as what medical practitioner usually does). This allows the device known as S³ (Smart Stethoscope) to be used at home without any special training or knowledge.

The apparatus is designed to be worn by the user/patient to allow continuous monitoring so as to be able to pick up abnormal heart sound signals even though the occurrence is non-regular or sporadic.

The apparatus improves of pre-diagnosis by highlighting to doctors of patient having early clues or signs of heart disease. The collected heart sound and ECG data will also be piped and stored on the cloud server for data analysis using machine learning. This is useful as it can perform pre-diagnosis classification of patients' heart condition. Abnormal heart sound segments of PCG data will be highlighted for doctor's review.

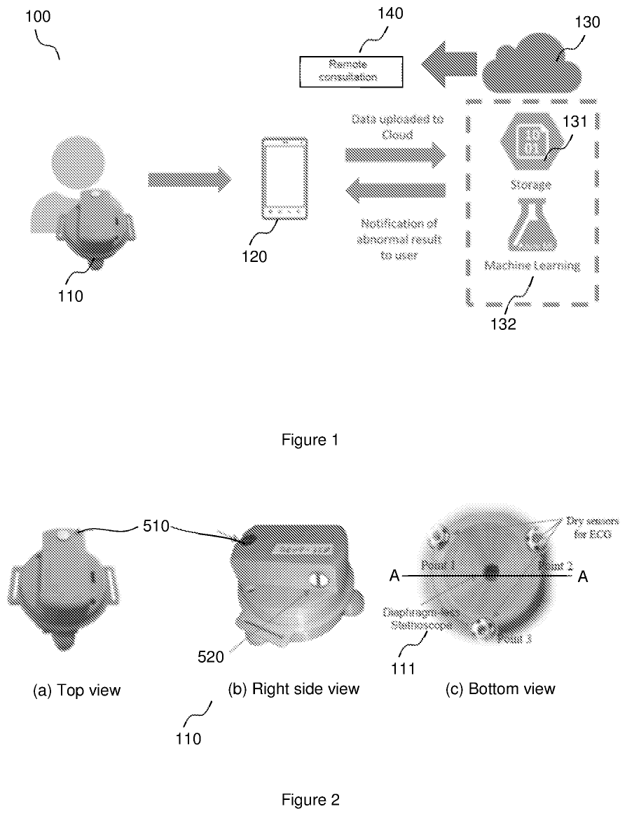

FIG. 1 shows the architecture of the system 100 implementing the apparatus.

The apparatus 110 is a wearable device communicatively connected to a mobile device 120. In response to receiving data from the apparatus 110, data in the mobile device 120 will be uploaded to cloud server 130 where the data would be stored on the database 131 and subsequently cleaned, analysed and classified using machine learning 132. The results can be viewed by doctors on cloud server 130. Any flagging on abnormal condition will result in notification being sent to doctors. In such event, doctor can schedule an early face-to-face appointment with the patient for further examination. The system 100 further includes an application installed on the mobile devices 120 of the patients. The application includes instructions to receive and transmit data among the apparatus 110, mobile device 120 and cloud server 130. Further details on the application will be described below.

The third heart sound (S3) may be the earliest clue to heart failure. It predicts a high risk of complications in non-heart surgery. The apparatus 110 is designed to be the frontline early detection in heart disease. Due to its ease of use it can be deployed in both home and healthcare centres.

Although there might be some high-end equipment that can replace the stethoscope, such equipment is not available in many rural areas. Furthermore, in paediatrics especially, X-rays are not recommended to reveal chest congestion and hence stethoscopes are still preferred for examination. Still further, only trained doctors who are experienced can perform diagnosis of an abnormal heart through the use of stethoscope. Therefore, the apparatus 110 is advantageous in that early detection of heart disease can be achieved without visiting a doctor. This in turn frees up the doctors to perform other medical services. Additionally, the apparatus 110 can also be applied to many applications beyond assisting in heart diagnosis, example detecting of lung anomaly, muscle degeneration, sign of life of a person, etc.

The system 100 is capable of continuous monitoring of the heart for electrical and mechanical activities via a mobile device; storage of data in the cloud; and an algorithm executable on the cloud server to detect heart anomalies.

The apparatus 110 is able to concurrently acquire electrocardiogram (ECG) and phonocardiogram (PCG) from the heart and can be configured as a wearable device for continuous monitoring (e.g. home monitoring) or as a digital stethoscope for remote consultation 140 (e.g. telemedicine). The PCG acquisition is based on a diaphragm-less design and ECG acquisition is based on dry electrodes. These are integrated into a hand-held configuration that can be placed or worn on the chest, near the heart. PCG signals acquired are not dependent on the location of the device as the algorithm is able to compensate for the variation of heart sound due to positional changes on the chest. Using the ECG as reference signal, the S1 and S2 of PCG are correctly identified. This enables the algorithm to identify S3 and/or S4 if present.

A mobile application is installed on the mobile device 120 to display the data acquired from the apparatus 110 and processed in cloud server 130. The mobile application may be based on Android, Windows 10, or iOS platform. The mobile application is also able to upload the signals to a data cloud server 130 for storage and/or processing. Further, this mobile application allows clinicians and/or individuals to view the ECG and PCG remotely, through access of data stored in the cloud server 130.

The algorithm performed by the cloud server 130 is configured to detect heart anomalies. The algorithm is capable of identifying the ECG signals and differentiates the heart sounds for effective processing. In addition, the algorithm is capable of self-learning to detect and determine the baseline for the individual and activate an alert when an anomaly is detected. In the event that the person being monitored needs immediate medical attention, an alert can be activated for the purpose of alerting the caregiver, clinician or designated individual if any immediate attention of intervention if required.

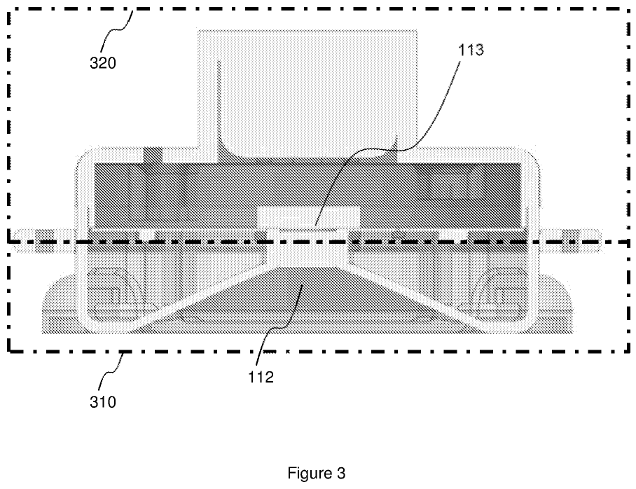

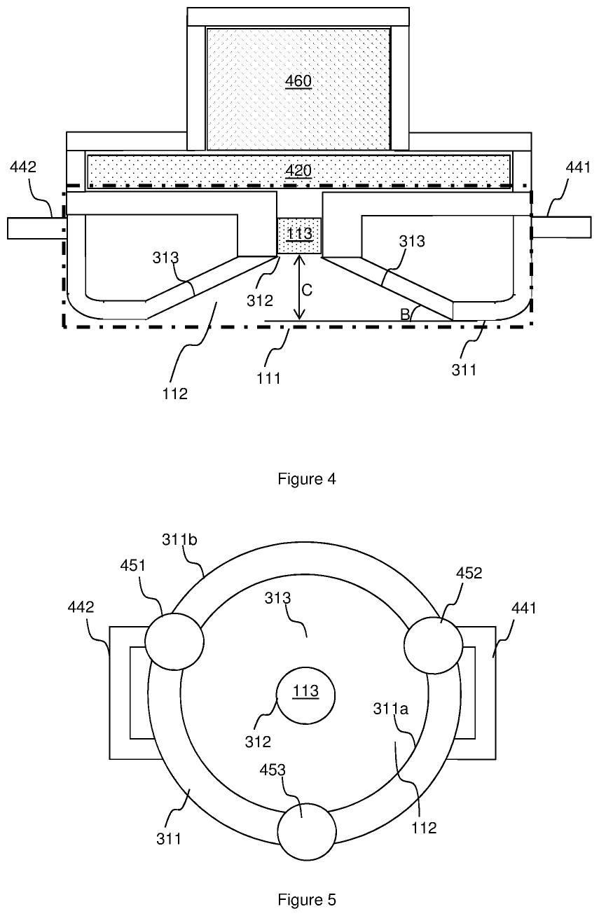

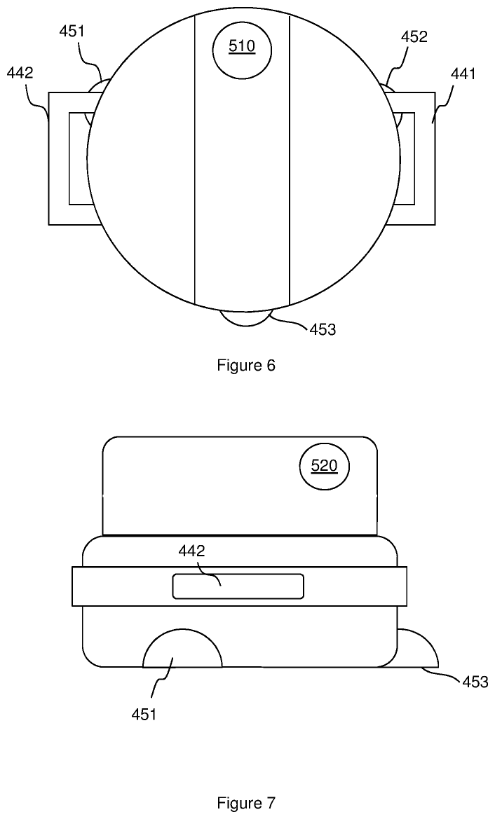

The apparatus 110 comprises an integrated ECG and PCG sensing platform with embedded electronics designed to achieve continuous monitoring of an individual. The apparatus 110 further comprises a network interface 420 in order to be communicatively connectable with the mobile device 120. FIG. 2 illustrates various perspective views of the exterior of the apparatus, namely, (a) top view, (b) right side view, and (c) bottom view. FIG. 3 illustrates a cross-sectional view of the apparatus 110. FIG. 4 illustrates a cross-sectional view between the line A-A as shown in the bottom view of the apparatus. FIG. 5 illustrates a bottom view of the apparatus 110. FIG. 6 illustrates a top view of the apparatus 110. FIG. 7 illustrates a right side view of the apparatus 110. The apparatus 110 comprises a housing having a bottom part 310 and a top part 320. The bottom part 310 comprises a diaphragm-less based stethoscope design 111 and acoustic chamber 112 for acquiring the PCG. The top part 320 comprises the battery and processing unit for processing the signal received from the microphone and dry electrodes.

The diaphragm-less based stethoscope design 111 decouples the need for a vibration media to pick up the heart sound. The acoustic chamber 112 is designed into the housing for electronics and is able to amplify the heart sound for acquisition (i.e. transducing to electrical signals) using microphone 113. The acoustic chamber 112 creates a snug fit around the chest and isolates the environmental noise to enable quality PCG signal acquisition. In addition, with the removal of the diaphragm, this design is able to remove noise generated by the abrasion of diaphragm against the shirt or skin when it moves. The acoustic chamber 112 is designed to ensure complete isolation for the electret capsule microphone from picking up any other acoustic noise. The microphone 113 is isolated at the opening 312 at the top of the acoustic chamber 112 with snug fit enabling vibration from within the chamber. For purposes of this description, the microphone 113 is an audio receiver that is able to convert sound into electrical signal. The audio receiver may include an analogue to digital converter to convert the electrical signal to digital signal.

The acoustic chamber 112 is tapered to an optimised angle between 23° to 25° B (between the bottom surface 311 of the bottom part 310 and the opening 312) to cater for noise isolation and various body types creating gap of minimum distance of about 7 to 9 mm from the microphone to the skin C. Specifically, a tapered surface 313 is provided between the bottom surface 311 of the bottom part 310 and the opening 312 forming the acoustic chamber 112. More specifically, the bottom surface 311 has an inner perimeter 311a and an outer perimeter 311b. The tapered surface 313 extends from the inner perimeter 311a to the perimeter of the opening 312 forming an acoustic chamber 112. Preferably, the acoustic chamber 112 is a conical shape. The microphone 113 is housed in the opening 312 and seals the opening 312. Within the acoustic chamber 112, no other cavities, other than the opening 112 which is completely sealed by the microphone 113, are visible to ensure noise isolation. This feature also helps in minimising the microphone 113 from picking up noises caused by motion artifact.

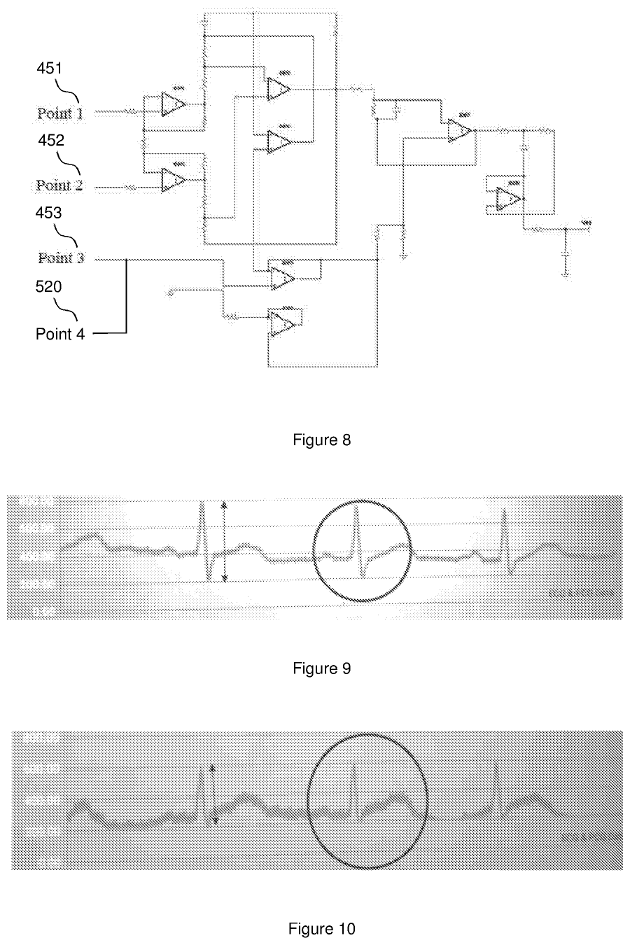

Three dry electrodes 451-453 are provided on the perimeter of the surface 311 to obtain ECG. The three dry electrodes 451-453 are arranged evenly apart from each other on the perimeter of the surface 311. When in use, the dry electrodes 451-453 are in contact with the skin of the subject. One skilled in the art will recognise that other types of dry sensors may be used for acquiring ECG signals without departing form the disclosure. For purposes of this description, dry electrodes are interchangeable with dry sensors. The use of dry sensors for acquiring ECG signal enables the device to be wearable. The configurations of these dry sensors, coupled with electronics circuit design as shown in FIG. 8, is uniquely designed to enable quality ECG signals acquisition within a small footprint.

To further enhance the ECG signal, the use of dry sensors 451-453 for acquiring ECG signal will be able to improve signal quality significantly with an additional dry sensor 520 provided on the side surface of the top part 320. Specifically, the dry sensor 520 is connected in parallel with the dry sensor 453 as shown in FIG. 8. FIGS. 9 and 10 show the different result obtained when using the same setup with and without touching dry sensor 520. When dry sensor 520 is in contact with the subject, the amplitude of the signal as shown in FIG. 9, is bigger compared to when the dry sensor 520 is not in contact with the subject as shown in FIG. 10. For purposes of the description, the subject places his/her finger in contact with the dry sensor 520 to provide stability to reference potential of ECG acquisition. Besides, setup with dry sensor connection is able to obtain a distinctive P wave, QRS complex and T wave (segment circled in FIG. 9) and has better Signal-to-Noise Ratio (SNR) compared to setup without dry sensors connection as shown in segment circled in FIG. 10. In brief, with the dry sensor 520, it is observed that the ECG data obtained from the dry sensors 451-453 has better Signal-to-Noise Ratio and hence, the peaks are more noticeable when compared to without the use of the dry sensor 520. The dry sensor 520 provides stability to reference potential of ECG acquisition, thus improves the quality of ECG signal.

Two attachment rings 441 and 442 may be provided on the side surface of the apparatus for strapping the apparatus 110 onto a subject.

The top part 320 of the housing comprises the battery 460 and processing unit 420 for processing the signal received from the microphone and dry electrodes. The top part 320 also includes a touch sensor 510 for activating the apparatus 110 as and when required.

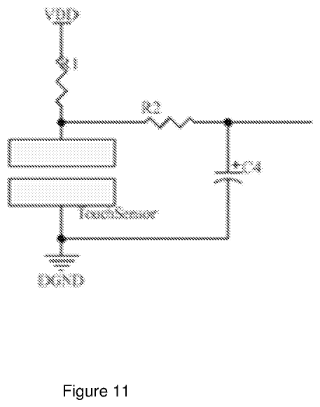

Touch sensor 510 is skin resistance based sensor. An exemplary use of this touch sensor designed for this apparatus is meant for user to activate the start of recording the ECG and PCG signals. The uniqueness in this sensor design is the use of skin resistance to generate a change in logic level to trigger an action. The sensor has no moving parts and consumes zero power when not touched by the user. It is a passive design and circuit working principle is to detect the change of resistance due to the presence of human skin. FIG. 11 illustrates an exemplary electronics circuit design of a touch sensor according to this disclosure.

Another dry electrode 520 is provided on the side surface of the top part 320. As mentioned above, the dry electrode 520 provides better SNR ECG data obtained from the dry electrodes 451-453.

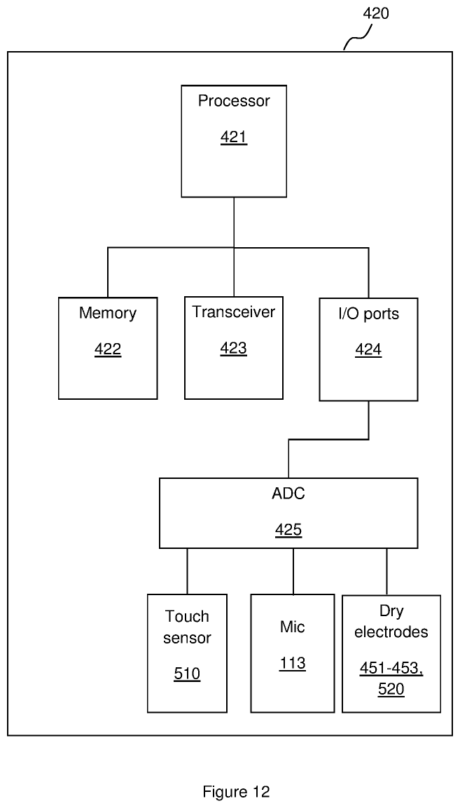

FIG. 12 illustrates the block diagram of an exemplary processing unit 420. The processing unit 420 can receive and transmit data, execute software applications. Processing unit 420 comprises a processor 421, memory 422, transceiver 423 and input/output ports 424.

The processor 421 is a processor, microprocessor, microcontroller, application specific integrated circuit, digital signal processor (DSP), programmable logic circuit, or other data processing device that executes instructions to perform the processes in accordance with the present invention. The processor 421 has the capability to execute various applications that are stored in the memory 422.

The memory 422 may include read-only memory (ROM), random-access memory (RAM), electrically erasable programmable ROM (EEPROM), flash cards, or any memory commonly used for computers.

The transceiver 423 is connected to an antenna which is configured to transmit outgoing data and receive incoming data over a radio communication channel. The radio communication channel can be a digital radio communication channel such as a WiFi, Bluetooth, RFID, NFC, DSRC, WiMax, CDMA, 3G/4G (or a future variant of cellular communication), GSM, or any other future wireless communication interface. Briefly, the transceiver 423 is required in order to be communicatively connectable with the mobile device 120.

One or more input/output (I/O) ports 424 can be configured to allow the processor 421 to communicate with and control from various I/O devices. Peripheral devices that may be connected to processing unit 420 via the I/O ports 424 include the circuitry shown in FIGS. 8 and 11, a USB storage device, an SD card or other storage device for transmitting information to or receiving information from the processing unit 420. In addition to updating applications stored on memory 422 or installing new applications onto the memory via the transceiver 423, a user may alternatively install new applications or update applications on the memory 422 through a user interface such as a USB via the I/O ports 424.

An analogue to digital converter (ADC) 425 may be provided to the processing unit 420 to convert the analogue signals from touch sensor 510, microphone 113 and dry sensors 451-453 and 520 to digital signals. The ADC 425 may be connected to one of the I/O ports 424. Alternatively, the ADC may be integrated to the I/O ports 424 without departing from the disclosure.

One skilled in the art will recognize that other features may be included in the processing unit 420. Further, the components in processing unit 420 may be replaced by other components that perform similar functions. In brief, the processing unit 420 as shown in FIG. 12 is considered merely illustrative and non-limiting.

In accordance with embodiments of this disclosure, instructions executable by the processor of processing unit 420 are stored in the memory 422. One skilled in the art will recognize that the instructions may be stored and/or performed as hardware, firmware, or software without departing from this disclosure.

All circuitries and processing unit 420 are powered by battery 460 directly or indirectly housed within the top part 320. The processing unit 420 is arranged between the battery 460 and the microphone 113 so that dry sensors, touch sensor, microphone, and circuitries as shown in FIGS. 8 and 11 can be efficiency connected to the processing unit 420.

The instructions stored on the memory 422 executable by the processor include:
1. receiving signal from touch sensor 510 and in response, initiate collection of ECG and PCG from the dry electrodes and microphone respectively;
2. receiving signals from dry electrodes and microphone and storing the signals from the dry electrodes as ECG signals and the signals from the audio receiver as PCG signals in the memory;
3. receiving a request to connect via the transceiver and in response, attempt to connect to requestor; and
4. transmitting the ECG and PCG signals on the memory to the requestor upon successful connection with the requestor.

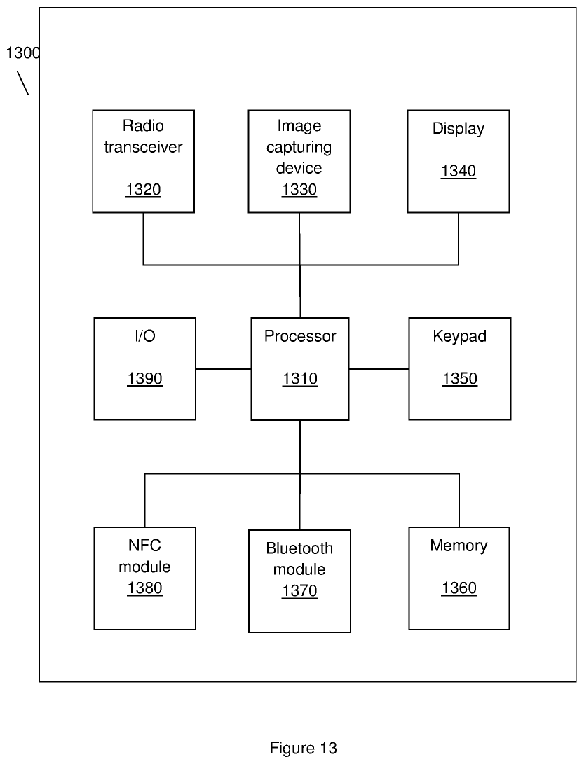

FIG. 13 illustrates an example of a processing system 1300 or a virtual machine running on the processing system 1300 of a mobile device 120 or a computing system performing as the cloud server 130. Particularly, processing system 1300 represents the processing systems in the mobile device 120 or a computing system performing as the cloud server 130 that execute instructions to perform the processes described below in accordance with embodiments of this disclosure. One skilled in the art will recognize that the instructions may be stored and/or performed as hardware, firmware, or software without departing from this invention. Further, one skilled in the art will recognize that the exact configuration of each processing system may be different and the exact configuration of the processing system executing processes in accordance with this invention may vary and processing system 1300 shown in FIG. 13 is provided by way of example only.

Processing system 1300 includes a processor 1310, a radio transceiver 1320, an image capturing device 1330, a display 1340, a keypad 1350, a memory 1360, a Bluetooth module 1370, a Near Field Communication (NFC) module 1380, and an I/O device 1390.

The radio transceiver 1320, image capturing device 1330, display 1340, keypad 1350, memory 1360, Bluetooth module 1370, NFC module 1380, I/O device 1390 and any number of other peripheral devices connect to processor 1310 to exchange data with processor 1310 for use in applications being executed by processor 1310.

The radio transceiver 1320 is connected to an antenna which is configured to transmit outgoing voice and data signals and receive incoming voice and data signals over a radio communication channel. The radio communication channel can be a digital radio communication channel such as a WiFi, Bluetooth, RFID, NFC, DSRC, WiMax, CDMA, 3G/4G (or a future variant of cellular communication), GSM, or any other future wireless communication interface.

The image capturing device 1330 is any device capable of capturing still and/or moving images such as complementary metal-oxide semiconductor (CMOS) or charge-coupled sensor (CCD) type cameras. The display 1340 receives display data from processor 1310 and display images on a screen for a user to see. The display 1340 may be a liquid crystal display (LCD) or organic light-emitting diode (OLED) display. The keypad 1350 receives user input and transmits the input to processor 1310. In some embodiments, the display 1340 may be a touch sensitive surface that functions as a keypad to receive user input.

The memory 1360 is a device that transmits and receives data to and from processor 1310 for storing data to a memory. The memory 1360 may include a non-volatile memory, such as a Read Only Memory (ROM), that stores instructions and data needed to operate various sub-systems of processing system 1300 and to boot the system at start-up. One skilled in the art will recognize that any number of types of memory may be used to perform this function. The memory 1360 may also include a volatile memory, such as Random Access Memory (RAM), that stores the instructions and data needed by processor 1310 to perform software instructions for processes such as the processes required for providing a system in accordance with this invention. One skilled in the art will recognize that any number of types of memory may be used as volatile memory and the exact type used is left as a design choice to those skilled in the art.

The Bluetooth module 1370 is a module that allows processing system 1300 to establish communication with another similar device such as processing unit 420 based on Bluetooth technology standard. The NFC module 1380 is a module that allows processing unit 1310 to establish radio communication with another similar device such as processing unit 420 by touching them together or by bringing the devices within a close proximity.

Other peripheral devices that may be connected to processor 1310 include a Global Positioning System (GPS) and other positioning transceivers.

The processor 1310 is a processor, microprocessor, or any combination of processors and microprocessors that execute instructions to perform the processes in accordance with the present disclosure. The processor has the capability to execute various application programs that are stored in the memory 1360. These application programs can receive inputs from the user via the display 1340 having a touch sensitive surface or directly from a keypad 1350. Some application programs stored in the memory 1360 that can be performed by the processor 1310 are application programs developed for UNIX, Android, IOS, Windows, Blackberry or other platforms.

The algorithm developed is able to automatically determine the heart sounds S1 and S2 and enables the apparatus 110 to be used without the need to know its position on the chest.

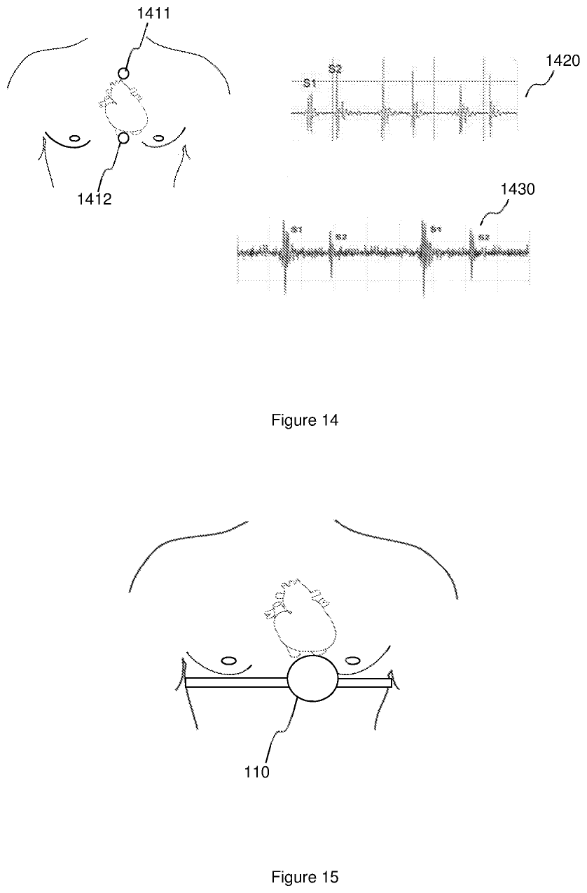

The use of stethoscope requires the knowledge of its position on the chest as illustrated in FIG. 14. Each position to acquire the heart sound will generate different signals as illustrated in the waveforms 1420 and 1430. For example, the base 1411 of heart is for acquiring sounds of aortic semilunar valve that are heard in second intercostal space at right sternal margin and pulmonary semilunar valve that are heard in second intercostal space at left sternal margin; and the apex 1412 of heart is for acquiring sounds of nitral valve that are heard over heart apex in the fifth intercostal space in line with middle of clavicle, and sounds of tricuspid valve that are typically heard in right sternal margin of fifth intercostal space (variations include over sternum or over left sternal margin in fifth intercostal space). In short, positioning the stethoscope at the base 1411 of heart would produce heart sound with dominance of S2 over S1 as shown in 1420 while position the stethoscope at apex 1412 of heart would produce heart sound with dominance of S1 over S2 as shown in 1430.

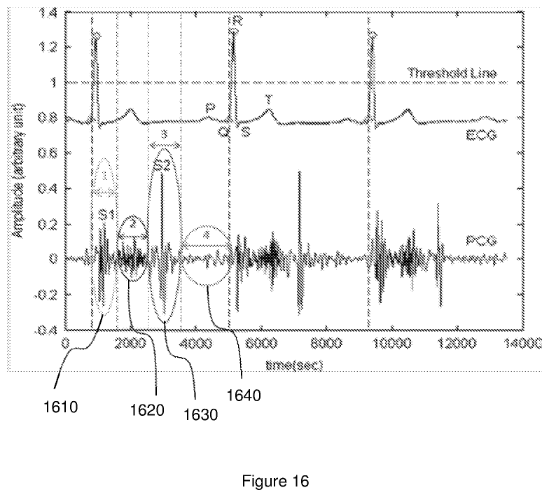

The algorithm developed for this disclosure is able to identify the heart sounds S1 and S2 regardless of the position of acquisition on the chest using ECG as the reference signal. FIG. 15 illustrates the apparatus 110 begin strapped to a chest of a subject. FIG. 16 illustrates an exemplary waveform acquired for signal processing where the apparatus is strapped to a subject as show in FIG. 15. As shown in FIG. 16, the identifications of S1 and S2 will also enable the marking of 4 regions (marked as 1, 2, 3 and 4) that will be used to determine if heart sound anomaly has occurred. The correctness in identifying heart sounds in each of these regions is crucial in the diagnosis of heart anomalies, which could be asymptomatic or event triggered.

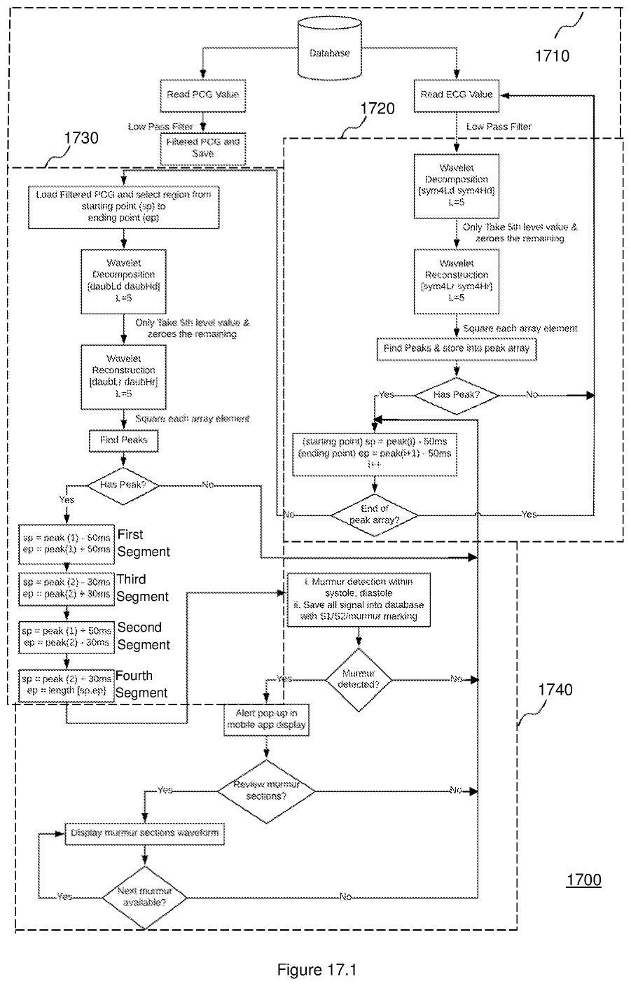

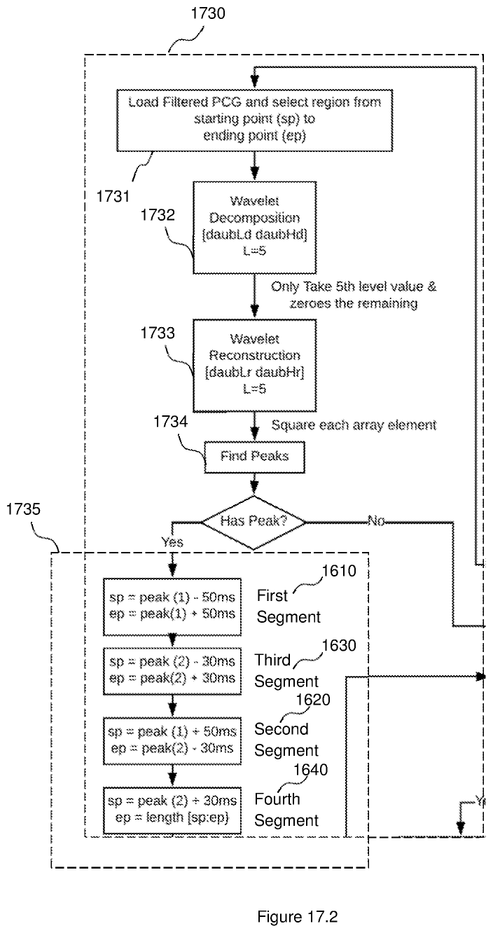

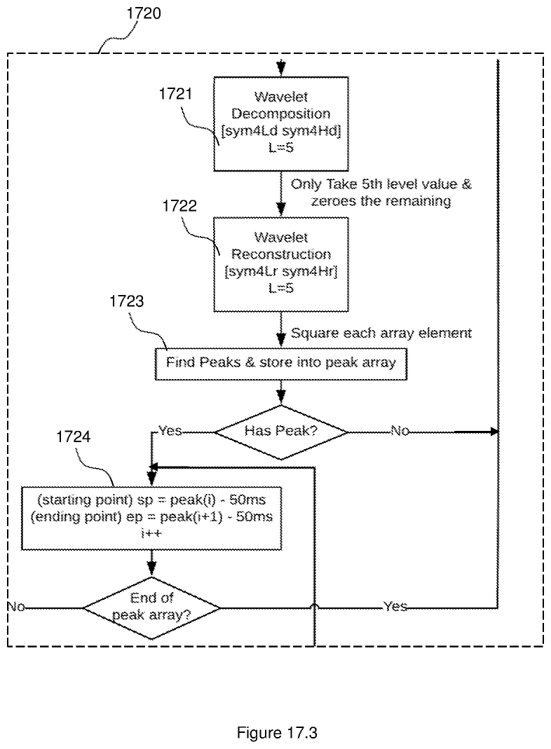

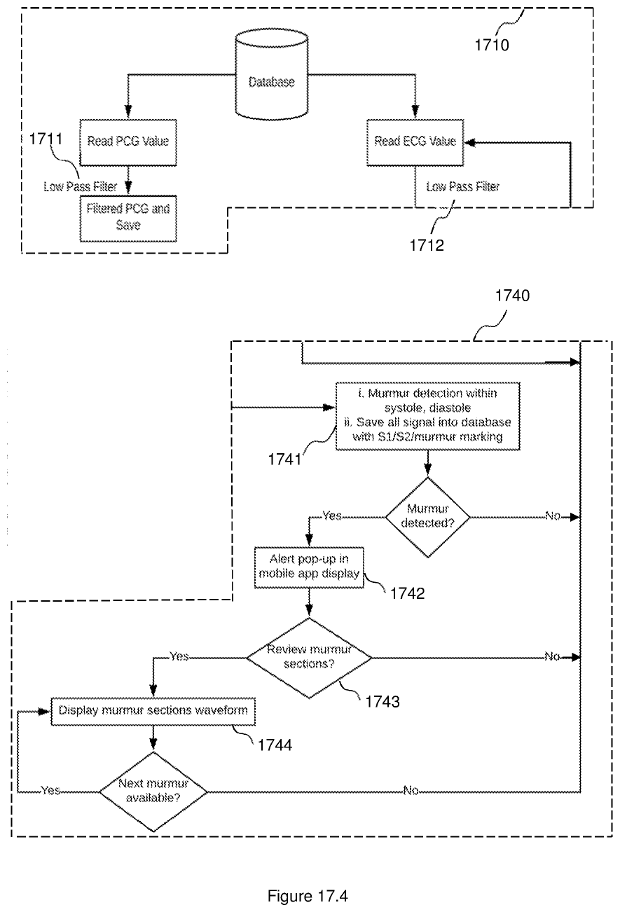

FIG. 17 illustrates an overview of the algorithm 1700 to process the ECG and PCG. As shown in FIG. 17, the first stage 1710 is to extract the PCG and ECG data for a particular patient and apply a low pass filter. The second stage 1720 is to analyse the ECG data to determine the starting point and ending point based on two consecutive peaks. In stage 3, a region of the filtered PCG data corresponding to the starting point and ending point determined of the ECG data is selected and the relevant peaks are marked accordingly. In the fourth stage 1740, the data is analysed and an alert is sent to the relevant mobile devices to alert of abnormal heart signals.

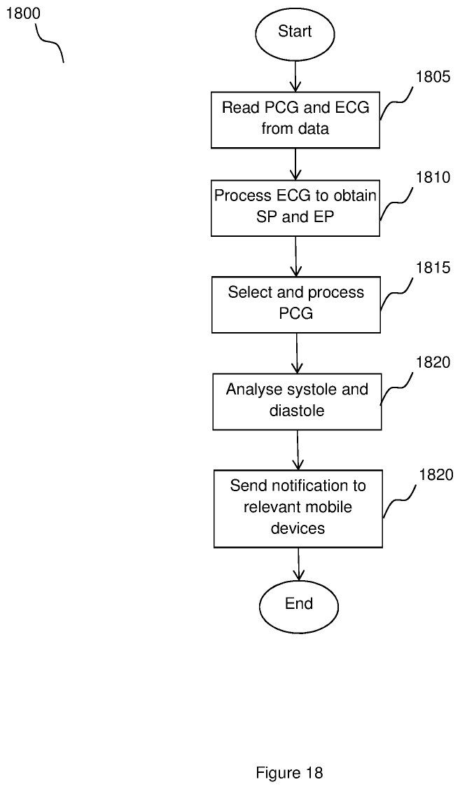

FIG. 18 illustrates a process flow 300 of the algorithm to process the ECG and PCG. Process 1800 starts step 1805 with the reading of the discrete signals from the database 131 where the signals were captured and stored. Respective low pass filter (as shown as 1711 and 1712 in FIG. 17.4) will be applied for each of the ECG and PCG signals to remove noise. Each of these signals are processed separately before the extracted data are used in combination to identify heart sound anomalies such as the systole murmur and diastole murmur from the PCG as follows.

In step 1810, process 1800 processes the filtered ECG signal. In particular, a wavelet decomposition (as shown as 1721 in FIG. 17.3) up to selected level is applied to the filtered ECG signal. The selected level is dependent on the selected sampling frequency being applied to sample the ECG signal. In the example as shown in FIG. 17.3, the selected level is the 5^{th} level. Specifically, a wavelet decomposition up to 5^{th} level (as shown as 1721 in FIG. 17.3), using wavelets such as Symlets 4 wavelets, is applied to the filtered ECG signal. Only the 5^{th} level value is taken while the remaining values are zeroed. By taking only the 5^{th} level analysis coefficients and putting the coefficients of all other levels to zero, the wavelet reconstruction (as shown as 1722 in FIG. 17.3) using the Symlets 4 wavelet was carried out to resynthesize the signal. By taking the absolute of the square values and identifying peaks within the signal, the R peaks of the ECG will thus be detected (as shown as 1723 in FIG. 17. The segment between two R-R peaks of ECG is then shifted left by a range of 50 ms to 150 ms to ensure PCG data before the first ECG peak is captured (as shown as 1724 in FIG. 17.3). For example, as shown in FIG. 17, the two R-R peaks of ECG are shifted left by 50 ms. Specifically, the starting point is defined as peak(i)-50 ms and ending point (EP) is defined as peak(i+1)-50 ms for i=1, 2, 3, . . . , n. In brief, the filtered ECG signal is processed in the following sequence:

1. apply a wavelet decomposition to the filtered ECG signal and zeroing all coefficients other than the selected level value (in the example shown in FIG. 17.3, the selected level is 5^{th} level);

2. apply a wavelet reconstruction to resynthesize the signal;

3. determine the R peaks of the ECG signal by taking the absolute of the square values;

4. shift the first and second R peaks values left by 50 ms; and

5. assign the first shifted R peak value as the SP and the second shifted R peak as the EP.

In step 1815, process 1800 selects the region between the SP and EP of the filtered PCG signal and analyses the PCG discrete signal of the selected region (as shown as 1731 in FIG. 17.2). In analysing the PCG discrete signal of the selected region, a wavelet decomposition up to is applied to the selected region of the filtered PCG signal. The selected level is dependent on the selected sampling frequency being applied to sample the PCG signal. In the example as shown in FIG. 17.2, the selected level is the 5^{th} level. Specifically, a wavelet decomposition up to 5th level, using wavelets such as Daubechies 4 wavelets, is applied to the PCG discrete signal of the selected region (as shown as 1732 in FIG. 17.2). By taking only the 5th level analysis coefficients and putting the coefficients all other levels to zero, the wavelet reconstruction using the Daubechies 4 wavelet was carried out to resynthesize the signal (as shown as 1733 in FIG. 17.2). By taking the absolute of the square values and identification of peaks within the signal, the S peaks of the PCG will thus be detected (as shown as 1734 in FIG. 17.2). In brief, the filtered PCG signal is processed in the following sequence:

1. apply a wavelet decomposition to the filtered PCG signal and zeroing all coefficients other than the selected level value (in the example shown in FIG. 17.2, the selected level is 5^{th} level);

2. apply a wavelet reconstruction to resynthesize the signal; and

3. determine the S peaks of the PCG signal by taking the absolute of the square values. After the S peaks of the PCG signal is determined, various segments 1610-1640 are marked and identified as described below.

Typical S1 duration is between 70 ms to 150 ms, and typical S2 duration is between 60 ms to 120 ms, the starting point (SP) and ending point (EP) of peak segment can be adjusted accordingly for each subject. As an example, each segment 1610-1640 is adjusted to have the following duration with respect to peaks as shown in 1735.

SP of Segment 1=peak(1)−50 msec

EP of Segment 1=peak(1)+50 msec

SP of Segment 3=peak(2)−30 msec

EP of Segment 3=peak(2)+30 msec

SP of Segment 2=peak(1)+50 msec

EP of Segment 2=peak(2)−30 msec

SP of Segment 4=peak(2)+30 msec

EP of Segment 4=EP of selected region

Based on the R peaks detected in the ECG, the first S peak of the PCG falls after it is taken as S1 peak, and the second S peak would be taken as S2 peak. This is how the QRS segment of the ECG is used to determine the location of S1 peaks and S2 peaks. Specifically, first segment 1610 in FIG. 16 relates to the first peak (the first segment contains the first heart sound (S1)), third segment 1630 in FIG. 16 relates to the second peak (the third segment contains the second heart sound (S2)), second segment 1620 in FIG. 16 relates to the region between the S1 and S2, and fourth segment 1640 in FIG. 16 relates to the region between S2 and the end of the selected region.

In step 1820, process 1800 analyses for murmur within second and fourth segments (as shown as 1741 in FIG. 17.4) in the following manner.

In order to determine heat sound anomaly such as the systole murmur, the frequency and the energy of the signal between S1 and S2 (second segment 1620 shown in FIG. 16) is calculated. If the frequency and energy of the signal are beyond a predetermined threshold, it is classified as abnormal and process 1800 proceeds to step 1820 and send a notification to the relevant mobile device in order to alert the relevant patient and/or doctor for further analysis.

In order to determine heart sound anomaly such as the diastole murmur, the frequency and the energy of the signal after S2 (fourth segment 1640 shown in FIG. 16) is calculated. If the frequency and energy of the signal were beyond a predetermined threshold, it is classified as abnormal and process 1800 proceeds to step 1820 and send a notification to the relevant mobile device in order to alert the relevant patient and/or doctor for further analysis.

A baseline may be determined for each patient in order to determine the predetermined threshold as mentioned above. For example, measurement of resting ECG and PCG are obtained over a predetermined period of time. This measurement will form the baseline and a threshold of certain percentage above the baseline is taken as the predetermined threshold when determining anomaly in step 1820. For example, the predetermined threshold for the second segment 1620 to determine systole murmur may be 20% above the baseline of second segment 1620; while the predetermined threshold for the fourth segment 1640 to determine diastole murmur may be 20% above the baseline of the fourth segment 1640. One skilled in the art will recognise that other method of setting a threshold may be implemented without departing from the disclosure. Additional steps may be provided after step 1820 is heart sound anomaly is detected (i.e. classification of abnormal heart sound within second and/or fourth segments 1620 and 1640. For example, if murmur is detected, an alert will be displayed to the user (as shown as 1742 in FIG. 17.4). If the user requests for review of the murmur segments (as shown as 1743 in FIG. 17.4), process 1800 displays the murmur segments waveform (as shown as 1744 in FIG. 17.4).

Process 1800 ends after step 1820. This process is iterated till all peaks are processed for each patient. Process 1800 may be performed on the mobile device 120 or the cloud server 130. If performed by the cloud server 130, the additional steps as described above in relation to alerting and displaying the murmur segments waveform would be transmitted to the mobile device 120 of the doctor. For example, an alert will be transmitted to the relevant mobile device 120.

The system 100 disclosed in this disclosure is able to deliver continuous monitoring of electrical and mechanical activities of the heart for detection of heart anomalies that could be event triggered or asymptomatic.

Comparing to existing digital stethoscope (e.g. 3M™ Littmann®, Thinklab One, etc) or ECG wearable devices (e.g. Spyder, Holter, etc), the system 100 is able to acquire both ECG and PCG concurrently for holistic assessment of the heart functionalities. The algorithm developed as part of the system 100 enables the use of our device as a wearable device where identification of heart sound S1 and S2 is not dependent on the device location.

In brief, the system comprises of the following:

a. An acoustic chamber designed for optimised acquiring of PCG with little or no noise artifacts.

b. Using an electret microphone which covers frequencies of 20 Hz20 kHz, the apparatus 110 is able to acquire PCG directly from the individual;

c. Acquisition of ECG using dry sensors in a small footprint, with the option to enhance the signal quality through an externally configured touched pad; and

d. Design of electronics to remove environmental noise and digitally process the data for wireless transmission to a mobile device.

In one embodiment, the software can process the signals using the algorithm developed and present the assessment outcome directly. Specifically, process 1800 may be provided as an application on the mobile device so that the patient or doctor is able to read the PCG and ECG signals directly in real time. In another embodiment, the application on the mobile device allows a patient or doctor to download the assessment outcome from the cloud server 130 and present it to the user.

An exemplary integration of apparatus, application on mobile device and algorithm on the cloud server as shown in FIG. 1 will enable remote consultation, continuous monitoring and caregiver alert when heart anomaly is detected.

The algorithm performed by the cloud server will enable data to be processed remotely and reduce the power consumption on the mobile device 120. The machine learning algorithm will be able to provide continuous assessment of the individual and develop personal baseline so that clinicians and caregivers will be alerted when there are significant deviations from these baselines. Additionally, population baselines can also be established and variations of individual ECG and PCG from these population baselines can also be used to trigger an alert to the pre-designated person.

### INDUSTRIAL APPLICATIONS

The system 100 can be used by clinicians for continuous monitoring of ECG and PCG for patients with asymptomatic heart anomaly. This is similar to the Holter monitoring, which only monitors the ECG whilst the system 110 according to this disclosure monitors both ECG and PCG concurrently for better medical diagnosis. In addition, clinicians can also use the system to conduct remote clinical assessment (e.g. tele-medicine) and provide effective assessments of heart anomaly.

In lifestyle application, an individual can use apparatus 110 to monitor his ECG and PCG for health assessment. In a home setup, caregivers can use apparatus 110 to monitor the ECG and PCG of their family and be alerted when heart anomaly occurs.

The above is a description of exemplary embodiments of a system and method for monitoring the heart based on ECG and PCG readings in accordance with this disclosure. It is foreseeable that those skilled in the art can and will design alternative systems and methods based on this disclosure.

1. An integrated electrocardiogram (ECG) and phonocardiogram (PCG) apparatus comprising:
a housing having a top part and a bottom part, the bottom part of the housing includes a tapered surface extending from a perimeter of a bottom surface to an opening at the top of the bottom part forming an acoustic chamber;
a power source housed within the top part;
an audio receiver arranged to seal the opening for obtaining PCG signal;
a plurality of dry sensors arranged at the bottom surface for obtaining ECG signal;
a processing unit powered by the power source and communicatively connectable to the audio receiver and the plurality of dry sensors, wherein the bottom part of the housing is adaptably configured to create a snug fit on a subject preventing the audio receiver from picking acoustic noise from outside the acoustic chamber.

2. The integrated ECG and PCG apparatus according to claim 1 wherein the audio receiver is an electret microphone which covers frequencies of 20 Hz˜20 kHz.

3. The integrated ECG and PCG apparatus according to claim 1 further comprising another dry sensor arranged at the top part of the housing and in parallel connection with one of the plurality of dry sensor.

4. The integrated ECG and PCG apparatus according to claim 1 further comprising a pair of attachment rings on a side surface of the housing.

5. The integrated ECG and PCG apparatus according to claim 1 further comprising a touch sensor for activating the processing unit.

6. The integrated ECG and PCG apparatus according to claim 5 wherein the touch sensor is skin resistance based sensor.

7. The integrated ECG and PCG apparatus according to claim 1 wherein the processing unit comprises: a processor, memory, transceiver, analogue to digital converter, and instructions stored on the memory and executable by the processor to:
receive signals from dry sensors and audio receiver and store the signals from the dry sensors as ECG signals and the signals from the audio receiver as PCG signals in the memory;
receive a request to connect via the transceiver and in response, attempt to connect to a requestor; and
transmit the ECG and PCG signals stored on the memory to the requestor upon successful connection with the requestor.

8. The integrated ECG and PCG apparatus according to claim 5 wherein the processing unit comprises: a processor, memory, transceiver, analogue to digital converter, and instructions stored on the memory and executable by the processor to:
receive signal from the touch sensor and in response, initiate collection of ECG and PCG signals from the dry sensors and audio receiver respectively;
receive signals from dry sensors and audio receiver and store the signals from the dry sensors as ECG signals and the signals from the audio receiver as PCG signals in the memory;
receive a request to connect via the transceiver and in response, attempt to connect to a requestor; and
transmit the ECG and PCG signals stored on the memory to the requestor upon successful connection with the requestor.

9. A heart monitoring system comprising:
the integrated ECG and PCG apparatus according to any one of claims 1-8;
a processing unit comprising a processor, memory and instructions stored on the memory and executable by the processor to:
receive the signal from the integrated ECG and PCG apparatus;
apply a low pass filter to each of the ECG and PCG signals;
process the filtered ECG signal to obtain a start point (SP) and an ending point (EP);
select a region between the SP and EP of the filtered PCG signal and analyse the PCG discrete signal of the selected region to determine a first segment, a second segment, a third segment and a fourth segment.

10. The heart monitoring system according to claim 9 wherein the instruction to process the filtered ECG signal to obtain a start point (SP) and an end point (EP) comprises instructions to:
apply a wavelet decomposition to the filtered ECG signal and zeroing all coefficients other than a selected level value;
apply a wavelet reconstruction to resynthesize the signal;
determine the R peaks of the ECG signal by taking the absolute of the square values;
shift the first and second R peaks values left by a predetermined range; and
assign the first shifted R peak as the SP and the second shifted R peak as the EP.

11. The heart monitoring system according to claim 10 wherein the instruction to analyse the PCG discrete signal of the selected region to determine a first segment, a second segment, a third segment and a fourth segment comprises instructions to:
apply a wavelet decomposition to the filtered ECG signal and zeroing all coefficients other than a selected level value;
apply a wavelet reconstruction to resynthesize the signal;
determine the S peaks of the PCG signal by taking the absolute of the square values; and
identify the first, second, third and fourth segments of the cardiac cycle which includes the first heart sound (S1) and second heart sound (S2) based on the detected S peaks of the PCG signal.

12. The heart monitoring system according to claim 11 wherein the instruction to identify the first, second, third and fourth segments of the cardiac cycle which includes the first heart sound (S1) and second heart sound (S2) based on the S peaks of the PCG signal comprises instructions to:
identify the first segment from −50 ms of the first S peak to +50 ms of the first S peak, the first segment containing the first heart sound (S1);
identify the third segment 3 from −30 ms of the second S peak to +30 ms of the second S peak, the third segment containing the second heart sound (S2);
identify the second segment from +50 ms of the first S peak to −30 ms of the second S peak; and
identify the fourth segment from +30 ms of the second S peak to end of selected region.

13. The heart monitoring system according to claim 12 further comprises instructions to:
determine heart sound anomaly based on the second segment;
determine heart sound anomaly based on the fourth segment; and
classify as abnormal in response to determining heart sound anomaly.

14. The heart monitoring system according to claim 13 wherein the instruction to determine heart sound anomaly based on the second segment comprises instructions to:
calculate frequency and energy from the second segment;
compare the frequency and energy with a predetermined threshold; and
classify as abnormal in response to the frequency and energy being above the predetermined threshold.

15. The heart monitoring system according to claim 13 wherein the instruction to determine heart sound anomaly based on the fourth segment comprises instructions to:
calculate frequency and energy from the fourth segment;
compare the frequency and energy with a predetermined threshold; and
classify as abnormal in response to the frequency and energy being above the predetermined threshold.
